(19) United States
(12) Patent Application Publication
(10) Pub. No.: US 2024/0238563 A1
(43) Pub. Date: Jul. 18, 2024

(54) CATHETER HOLDER

(71) Applicant: Terumo Kabushiki Kaisha, Tokyo (JP)

(72) Inventors: Takahito ARAKI, Saitama city (JP); Takashi KITAOKA, Hadano city (JP)

(73) Assignee: Terumo Kabushiki Kaisha, Tokyo (JP)

(21) Appl. No.: 18/413,854

(22) Filed: Jan. 16, 2024

(30) Foreign Application Priority Data
Jan. 16, 2023 (JP) 2023-004737

(51) Int. Cl. A61M 25/02

(52) U.S. Cl. CPC A61M 25/02; A61M 2025/0213; A61M 2025/0266; A61M 2025/028

(57) ABSTRACT

A catheter holder capable of stably holding a catheter on a body surface of a patient by facilitating catheter routing when the catheter is mounted on the body surface includes first and second components. The first component has a base portion configured to adhere to the patient's body surface and a receiving portion configured to receive a portion of a catheter disposed inside the patient's body and extending to outside of the body. The second component has a holding portion configured to hold the catheter and/or a connector connected to the catheter, with the second component being rotatably connected to the first component. The first or second component includes a catheter guide portion configured to bend the catheter, which extends from the receiving portion, toward the holding portion, and the catheter guide portion is formed with a crush prevention portion configured to prevent the catheter from being crushed.

### CROSS-REFERENCES TO RELATED APPLICATIONS

This application claims priority to Japanese Patent Application No. 2023-004737 filed on Jan. 16, 2023, the entire content of both of which is incorporated herein by reference.

### TECHNOLOGICAL FIELD

The present invention generally relates to a catheter holder for stably holding a catheter on a body surface of a patient.

### BACKGROUND DISCUSSION

When, for example, cerebral infarction develops as a brain disease, blood flow that supplies oxygen to brain cells is blocked, and the brain cells may be damaged. Therefore, when the cerebral infarction develops, early reperfusion of the blood flow is required. However, a proportion of patients who satisfy conditions for receiving a super-acute phase treatment (for example, tissue plasminogen activator (t-PA) administration or mechanical thrombectomy) with a high level of evidence currently performed in clinical practice, is low. Therefore, many patients can only select a conservative treatment.

One proposed treatment for cerebral infarction involves injecting a high oxygen solution such as an oxygenated cerebrospinal fluid into a medullary cavity of a patient and directly supply oxygen to brain cells in which oxygen is deficient. Here, U.S. Pat. No. 4,686,085B discloses a method and a system for providing an early stroke treatment. In the method and the system disclosed in U.S. Pat. No. 4,686,085, a nutrient emulsion is collected from cisterna magna through a conduit having a distal portion inserted near the cisterna magna. The oxygenated nutrient emulsion is injected into a lateral ventricle through an infusion cannula attached to a skull via a holder.

A catheter body surface fixing device for fixing such a catheter to a body surface of a patient is presented. An example is disclosed in Japanese Patent Application Publication No. 2003-062083 (JP2003-062083A).

The catheter body surface fixing device includes a portion that holds the catheter drawn out to the body surface perpendicularly to the body surface, a portion that holds the catheter with an inclination, and a portion that holds the catheter in a gentle arc shape and allows the portion that holds the catheter perpendicularly to communicate with the portion that holds the catheter with an inclination.

In a treatment of injecting the high oxygen solution into the medullary cavity of the patient, it is necessary to use a catheter made of a harder material that has higher pushability than that of a silicone catheter in order to quickly pass through the medullary

However, when the catheter body surface fixing device in the related art is used, there is no degree of freedom when a catheter made of a hard material is routed.

In addition, even when the catheter body surface fixing device disclosed in Japanese Patent Application Publication No. 2003-062083 is used, when a catheter is introduced from the catheter body surface fixing device to a puncture portion in lumbar vertebrae, the catheter may be bent at a substantially right angle in the vicinity of the puncture portion in the lumbar vertebrae to cause a kink.

Therefore, in the catheter body surface fixing device in the related art, there is a problem that it is difficult to prevent the catheter from kinking during a procedure.

### SUMMARY

Disclosed here is a catheter holder capable of stably holding a catheter on a body surface of a patient by improving a degree of freedom of routing when the catheter is mounted on the body surface.

Disclosed here is (1) a catheter holder including: a first component including a base portion configured to adhere to a body surface of a patient and a receiving portion configured to receive a portion of a catheter disposed in an inside of a body of the patient that extends to an outside of the body; and a second component including a holding portion configured to hold the catheter and/or a connector connected to the catheter, the second component being rotatably connected to the first component, in which the first component includes a catheter guide portion configured to bend the catheter, which extends from the inside of the body of the patient, toward the holding portion, and the catheter guide portion is formed with a crush prevention portion configured to prevent the catheter from being crushed.

According to the above configuration, the catheter holder includes the first component that can be fixed by adhering to the body surface of the patient, and the second component that is rotatably connected to the first component.

The catheter guide portion that bends the catheter extending from the receiving portion of the first component toward the holding portion of the second component is included. The catheter guide portion is formed with the crush prevention portion configured to prevent the catheter from being crushed.

Accordingly, even when it is necessary to use a catheter made of a material harder than that of a silicone catheter, a direction in which the catheter is disposed on the body surface can be freely changed according to a procedure, and when the catheter is extended from the inside of the body along the body surface, the crush prevention portion can prevent the catheter from being crushed, thereby preventing the catheter from kinking.

Therefore, the catheter holder can stably hold the catheter on the body surface of the patient by improving a degree of freedom of routing when the catheter is mounted on the body surface.

(2) In the catheter holder according to the above (1), preferably, the first component includes a central portion to which the second component is rotatably connected, and the base portion formed around the central portion and is adherable to the body surface, and the catheter guide portion is formed in the central portion.

According to the above configuration, the central portion of the first component of the catheter holder allows a rotary movement of the second component, the base portion is adherable to the body surface, and the catheter guide portion is formed in the central portion. Therefore, the first component can bend the catheter that extends from the catheter guide portion or the receiving portion toward the holding portion while adhering to and being fixed to the body surface of the patient.

Accordingly, even when it is necessary to use a catheter made of a material harder than that of the silicone catheter, the catheter can be more stably held on the body surface by improving the degree of freedom of routing when the catheter is mounted and by adhering the catheter holder to the body surface.

(3) In the catheter holder according to the above (1) or (2), preferably, the base portion includes an adhesive portion in close contact with the body surface of the patient.

According to the above configuration, the adhesive portion of the base portion of the catheter holder brings the catheter holder into close contact with the body surface. Therefore, the catheter can be more stably held on the body surface even when it is necessary to use a catheter made of a material harder than that of the silicone catheter.

(4) The catheter holder according to any one of the above (1) to (3) preferably includes: a plurality of the base portions, in which the base portions preferably extend in different directions from the central portion.

According to the above configuration, the base portions of the catheter holder extend in different directions from the central portion. Therefore, the base portions can stably fix the first component to the body surface of the patient in a state where the first component adheres to the body surface, even when it is necessary to use a catheter made of a material harder than that of the silicone catheter.

(5) In the catheter holder according to any one of the above (1) to (4), preferably, the second component includes a mounting portion detachably mounted on the central portion of the first component, and the central portion is connected to the mounting portion of the second component such that the mounting portion of the second component is rotatable about the central portion.

According to the above configuration, the mounting portion of the second component of the catheter holder is connected to be rotatable about the central portion of the first component. Therefore, the second component can be rotated and positioned at any position around the central portion of the first component, and a disposition direction of the catheter can be freely changed according to the procedure.

(6) In the catheter holder according to any one of the above (1) to (5), preferably, the catheter guide portion includes a catheter holding groove portion into which an intermediate portion of the catheter is fitted and positioned and held in the central portion.

According to the above configuration, a position of the catheter can be easily fixed simply by fitting the intermediate portion of the catheter into the catheter holding groove portion of the catheter holder, even when it is necessary to use a catheter made of a material harder than that of the silicone catheter.

(7) In the catheter holder according to the above (6), preferably, a plurality of the catheter holding groove portions are provided in the central portion, and the catheter holding groove portions are provided at different angles with respect to a center line of the central portion.

According to the above configuration, the catheter holding groove portions of the catheter holder are provided at different angles around a central axis of the central portion. Therefore, the disposition direction of the catheter can be changed according to the procedure simply by selecting the catheter holding groove portion at any angle and fitting the catheter, even when it is necessary to use a catheter made of a material harder than that of the silicone catheter.

(8) In the catheter holder according to the above (6), preferably, a depth of the catheter holding groove portion is equal to or larger than an outer diameter of the catheter.

According to the above configuration, it is possible to ensure a state where the catheter is embedded in any of the catheter holding groove portions of the catheter holder, and thus it is possible to avoid a risk that the catheter is crushed due to an external factor.

(9) In the catheter holder according to any one of the above (1) to (5), preferably, the holding portion is a concave portion into which the catheter is detachably fittable to be held.

According to the above configuration, even when it is necessary to use a catheter made of a material harder than that of the silicone catheter, the catheter can be easily and reliably held by being freely detachably fitted into the holding portion that is the concave portion of the catheter holder.

(10) The catheter holder according to the above (5) preferably includes: a fitting portion configured to rotatably connect the mounting portion of the second component to the central portion of the first component, in which the fitting portion preferably includes a concave portion and a convex portion fitted into the concave portion.

According to the above configuration, the fitting portion of the catheter holder can reliably connect the second component to the first component in a rotatable manner with a simple structure simply by fitting the convex portion into the concave portion.

(11) In the catheter holder according to the above (10), preferably, the fitting portion includes a groove portion as the concave portion formed around the central portion of the first component, and a projection portion as the convex portion formed in the mounting portion of the second component and fitted into the groove portion.

According to the above configuration, the second component can be reliably connected to the first component in a rotatable manner with a simple structure by forming the concave portion of the catheter holder around the central portion of the first component, and forming the convex portion on the mounting portion of the second component.

(12) In the catheter holder according to the above (5), preferably, the mounting portion of the second component includes a C-shaped portion having a C shape, and by fitting the C-shaped portion into the central portion of the first component, the second component is connected so as to be rotatable about the central portion of the first component.

According to the above configuration, the second component can be reliably connected to the first component in a rotatable manner with a simple structure simply by fitting the C-shaped portion of the second component into the central portion of the first component.

(13) In the catheter holder according to the above (1), preferably, an opening portion configured to receive the catheter that extends from the body surface of the patient to the outside of the body is provided at a center of the receiving portion, the receiving portion has a cutout open portion at an outer edge portion, and the opening portion and the open portion communicate with each other.

(14) In the catheter holder according to any one of the above (1) to (12), preferably, the catheter includes a supply catheter configured to supply a treatment liquid to a target lesion of the patient, a discharge catheter disposed coaxially with the supply catheter and configured to discharge the liquid from the target lesion to the outside of the body, and an interlock component configured to interlock the supply catheter and the discharge catheter, and the holding portion of the second component detachably fits and holds a part of the catheter and the interlock component.

According to the above configuration, the holding portion of the second component of the catheter holder can detachably hold the supply catheter, the discharge catheter, and the interlock component simply by fitting the supply catheter, the discharge catheter, and the interlock component into the holding portion. Therefore, the catheter and the interlock component can be easily attached and detached.

(15) In the catheter holder according to the above (14), preferably, the catheter is a spinal subarachnoid catheter.

According to the above configuration, in the procedure using the spinal subarachnoid catheter, the catheter holder can adhere to the body surface by improving the degree of freedom of routing when the catheter is mounted, and the catheter can be more stably held even when it is necessary to use a catheter made of a material harder than that of the silicone catheter.

The disclosure here involves (16) a catheter holder including: a first component including a receiving portion configured to receive a portion of a catheter disposed in an inside of a body of a patient that extends to an outside of the body; and a second component including a holding portion configured to hold the catheter and/or a connector connected to the catheter, the second component being rotatably connected to the first component, in which the first component or the second component includes a catheter guide portion configured to bend the catheter, which extends from the receiving portion, toward the holding portion, and the catheter guide portion is formed with a crush prevention portion configured to prevent the catheter from being crushed.

According to the above configuration, the catheter holder includes the first component and the second component rotatably connected to the first component.

The catheter guide portion that bends the catheter extending from the receiving portion of the first component toward the holding portion of the second component is included. The catheter guide portion is formed with the crush prevention portion configured to prevent the catheter from being crushed.

Accordingly, even when it is necessary to use a catheter made of a material harder than that of the silicone catheter, a direction in which the catheter is disposed on the body surface can be changed according to the procedure, and the crush prevention portion can prevent the catheter from being crushed, thereby preventing the catheter from kinking.

Therefore, the catheter holder can stably hold the catheter on the body surface of the patient by improving a degree of freedom of routing when the catheter is mounted on the body surface.

Another aspect of the disclosure involves a catheter holder in combination with a catheter to be mounted on a body of a patient to hold the catheter when the catheter is disposed inside the patient's body and extends to outside of the patient's body, wherein the catheter holder comprises first and second components. The first component includes a receiving portion and a body surface fixing base portion, with the body surface fixing base portion projecting away from the receiving portion as seen in plan view and the body surface fixing base portion being configured to be fixed to a body surface of the patient's body. The receiving portion has a surface configured to face the body surface of the patient when the body surface fixing base portion is fixed to the body surface of the patient's body. The receiving portion includes a through hole that extends through the receiving portion so that the through hole intersects the surface of the receiving portion, wherein the through hole in the receiving portion is configured to receive a portion of the catheter passing through the through hole and extending outside the patient's body. The second component includes a mounting portion and a holding portion, wherein the holding portion projects away from the mounting portion as seen in the plan view and is configured to hold the portion of the catheter extending outside the patient's body. The mounting portion is mounted on the receiving portion by virtue of the receiving portion and the mounting portion including engaging parts that are in engagement with one another in a manner that allows the mounting portion to be rotated relative to the receiving portion. The receiving portion of the first component includes a crush prevention portion constituted by a curved circumferential portion that merges with the through hole so that the portion of the catheter passing through the through hole passes over the curved circumferential portion and is led to the holding portion of the second component.

The catheter holder disclosed here has an advantage that it is capable of stably holding a catheter on a body surface of a patient by improving a degree of freedom of routing when the catheter is mounted on the body surface.

### BRIEF DESCRIPTION OF THE DRAWINGS

FIG. 1 is a block diagram showing an overview of a brain disease treatment system in which a catheter holder according to a first embodiment is used.

FIGS. 2A and 2B are diagrams showing a usage example of the catheter holder.

FIG. 3 is an exploded perspective view showing a structural example of the catheter holder.

FIG. 4 is a plan view showing the catheter holder in an assembled state.

FIG. 5 is a plan view showing the catheter holder in an exploded state.

FIG. 6 is a longitudinal cross-sectional view showing a vicinity of a crush prevention portion in a central portion.

FIG. 7 is a diagram showing an example of a normal use mode in which a catheter is abruptly bent by 90 degrees and introduced into an insertion port in a patient as a comparative example with respect to the first embodiment.

FIG. 8 is an exploded perspective view showing a second embodiment.

### DETAILED DESCRIPTION

Hereinafter, preferred embodiments of the catheter holder disclosed here will be described in detail with reference to the drawings.

The embodiments described below are preferred examples of the catheter holder, though the scope of the invention is not limited to these embodiments unless specifically specified in the following description. In the drawings, similar components of different embodiments are denoted by the same reference signs, and a detailed description of such components is not repeated.

### First Embodiment

### Overview of Brain Disease Treatment System 1 Using Catheter Holder 10

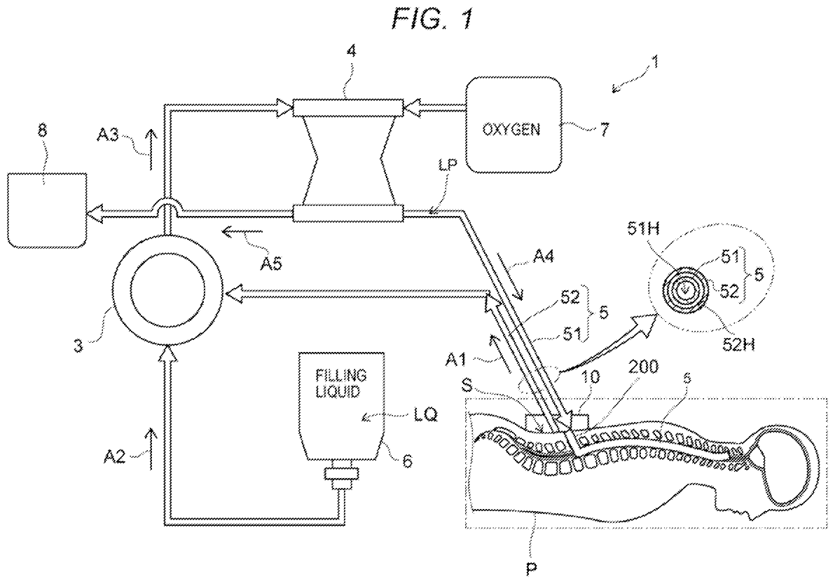

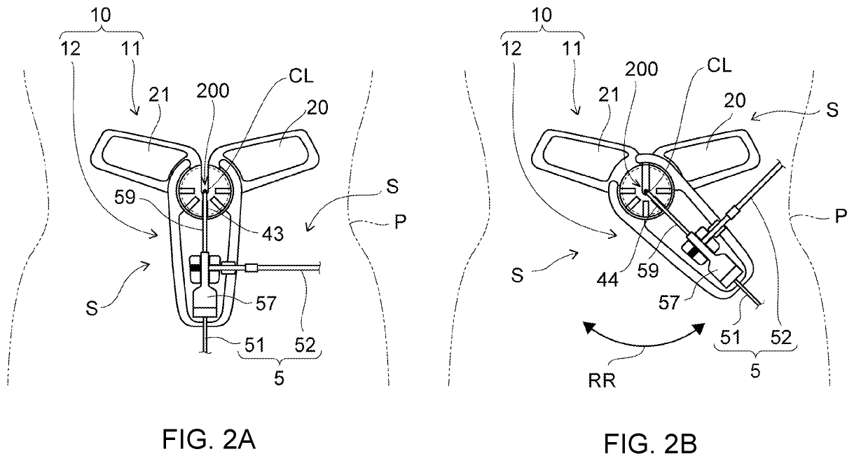

FIG. 1 is a block diagram showing an overview of the brain disease treatment system 1 in which the catheter holder (catheter body surface fixing device) 10 for fixing a catheter according to a first embodiment to a body surface is used. FIGS. 2A and 2B show a usage example of the catheter holder 10.

As shown in FIG. 1, the brain disease treatment system 1 is an example of a medical system in which the catheter holder 10 may be used.

The brain disease treatment system 1 includes a spinal subarachnoid catheter 5, a pump system 3, and an oxygenation mechanism 4. The catheter holder 10 is used at the spinal subarachnoid catheter 5.

The spinal subarachnoid catheter 5 is inserted into a subarachnoid space from, for example, a vicinity of lumbar vertebrae of a patient P, and a distal portion of the spinal subarachnoid catheter 5 is delivered to a target lesion as far as possible, for example, to cisterna magna.

The brain disease treatment system 1 injects a high oxygen solution (in the present embodiment, for example, an oxygenated cerebrospinal fluid) into a cerebrospinal fluid (CSF) (an example of a treatment liquid) present in the subarachnoid space, and sucks the cerebrospinal fluid present in the subarachnoid space and discharges the cerebrospinal fluid to an outside of a body of the patient P.

The pump system 3 shown in FIG. 1 is, for example, a centrifugal pump whose operation is controlled by a controller.

The pump system 3 sucks the cerebrospinal fluid in an inside of the body of the patient P via the spinal subarachnoid catheter 5 as indicated by an arrow A1.

The pump system 3 sucks a filling liquid LQ stored in a storage tank 6 as indicated by an arrow A2 as necessary.

Then, the pump system 3 sends the cerebrospinal fluid sucked from the patient P to an oxygenation mechanism 4 side as indicated by an arrow A3.

Examples of the filling liquid LQ include, but are not particularly limited to, an artificial cerebrospinal fluid (aCSF) such as lactated Ringer.

The oxygenation mechanism 4 shown in FIG. 1 includes an oxygen supply source 7, and oxygenates the cerebrospinal fluid discharged from the subarachnoid space by supplying oxygen from the oxygen supply source 7 to produce a high oxygen solution LP.

The pump system 3 sucks the high oxygen solution LP supplied from the oxygenation mechanism 4 and injects the high oxygen solution LP into the cerebrospinal fluid in the subarachnoid space of the patient P via the spinal subarachnoid catheter 5, as indicated by an arrow A4.

The excess high oxygen solution LP may be discharged from the oxygenation mechanism 4 to a discharge tank 8 side as indicated by an arrow A5.

The pump system 3 performs injection and discharge with one pump as shown in FIG. 1, but in addition to this method, injection and discharge may be performed by a method of using two pumps, i.e., a discharge pump and an injection pump, a method of using an injection-only pump and allowing natural discharge, conversely, a method of using a discharge-only pump and allowing natural injection, or a method of performing injection and discharge by using potential energy such as an intravenous drip without using a pump.

In the present embodiment, the oxygenation mechanism 4 may be of a type using oxygen bubbling, or may be of a type in which a hollow fiber is immersed in the cerebrospinal fluid, oxygen is allowed to pass through the hollow fiber, and the cerebrospinal fluid is oxygenated through pores in a surface of the hollow fiber.

As shown in FIG. 1, catheters 51 and 52 of the spinal subarachnoid catheter 5 are inserted into the subarachnoid space from a vicinity of lumbar vertebrae S of the patient P.

A distal portion of the catheter 51 of the spinal subarachnoid catheter 5 or an opening portion for taking in and out a fluid is present, for example, in the subarachnoid space. As indicated by the arrow A4, the high oxygen solution LP is sent to the distal portion of the spinal subarachnoid catheter 5 and injected into the cerebrospinal fluid present in the subarachnoid space.

### Example of Cross-sectional Structure of Spinal Subarachnoid Catheter 5

As shown in FIG. 1, the spinal subarachnoid catheter 5 has a double-tube structure (co-axial structure) and includes the tubular injection catheter 51 (supply catheter) as a first tube and the tubular discharge catheter 52 (discharge catheter) as a second tube. The injection catheter 51 can be disposed in a lumen of the discharge catheter 52.

The injection catheter 51 is an inner tube having a lumen 51H, and the generated high oxygen solution LP is sent into the subarachnoid space of the patient P. The discharge catheter 52 is an outer tube having a lumen 52H, and collects and discharges the cerebrospinal fluid in the inside of the body of the patient P.

### Structural Example of Catheter Holder 10

Next, a preferred structural example of the catheter holder 10 will be described with reference to the drawings.

FIGS. 2A and 2B show states where the catheter 51 and the catheter 52 inserted into an insertion port 200 in the patient P are held in a state of extending to the outside of the body through the insertion port 200 by using the catheter holder 10.

The insertion port 200 is an example of a puncture portion provided in the lumbar vertebrae S of the patient P. In an example of a holding state of the catheter shown in FIG. 2A and in an example of a holding state of the catheter shown in FIG. 2B, the catheter holder 10 holds the catheter 51, the catheter 52, and a connector 57 of the spinal subarachnoid catheter 5 in different directions.

### First Component 11 and Second Component 12

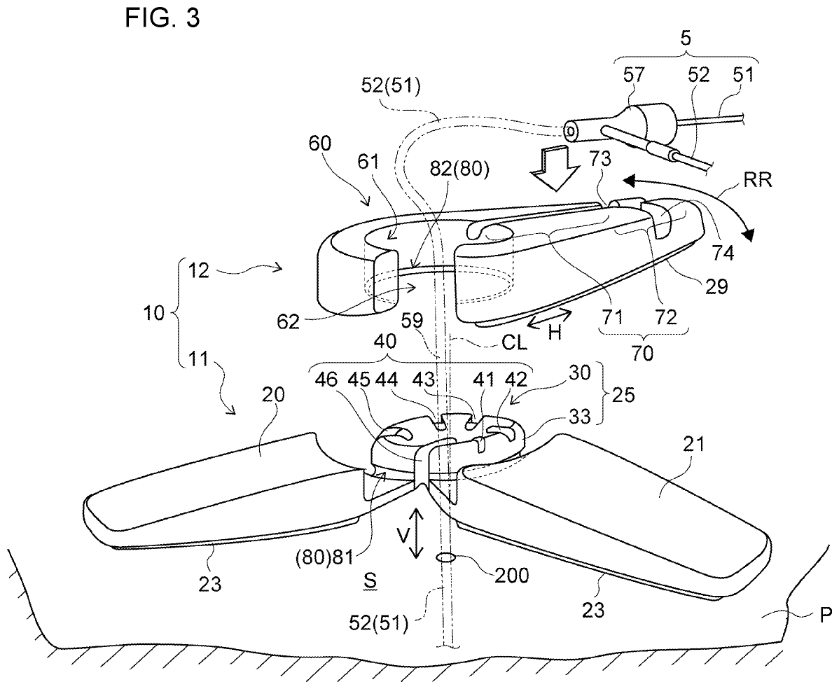

FIG. 3 is an exploded perspective view showing a structural example of the catheter holder 10.

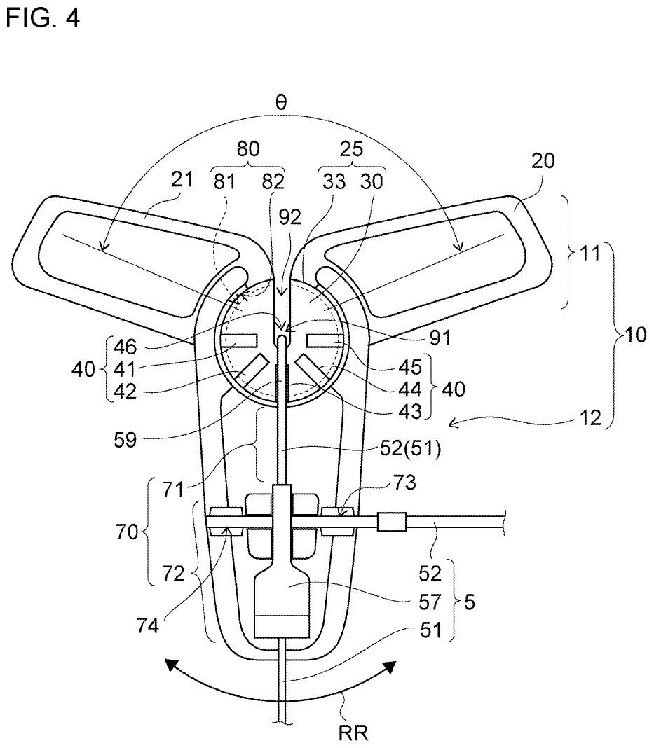

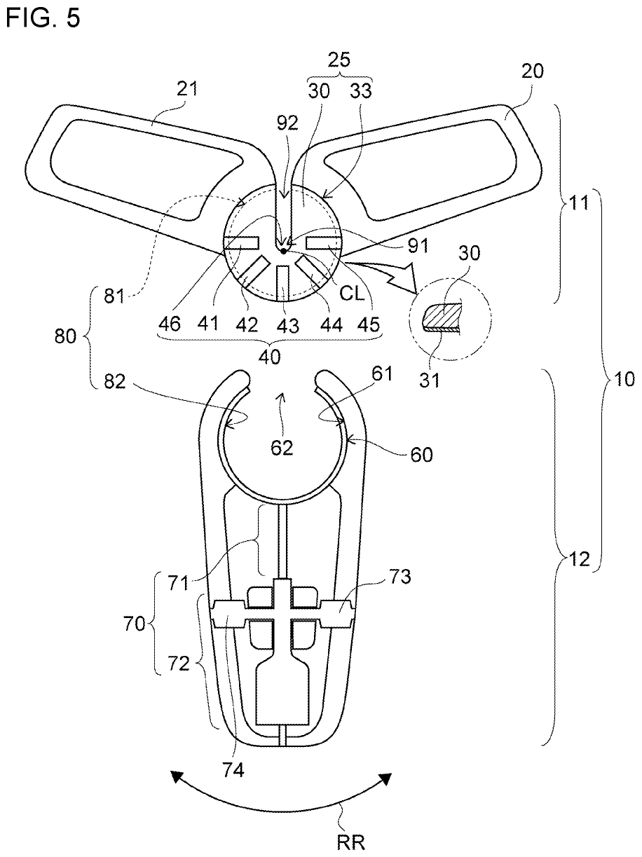

FIG. 4 is a plan view showing the catheter holder 10 in an assembled state. FIG. 5 is a plan view showing the catheter holder 10 in an exploded state.

As shown in FIGS. 3 to 5, the catheter holder 10 includes the first component 11 and the second component 12. The first component 11 and the second component 12 are both made of, for example, a lightweight resin that has little influence on a living body of the patient P.

As shown in FIGS. 3 and 4, the first component 11 preferably includes a plurality of base portions 20 and 21 and a receiving portion 25. The base portions 20 and 21 are portions (body surface fixing portions) that detachably adhere to, for example, a body surface for the lumbar vertebrae S of the patient P.

The receiving portion 25 is a portion that receives and holds portions of the catheters 51 and 52 disposed in the inside of the body of the patient P that extend to the outside of the body.

The receiving portion 25 includes, at a center thereof, an opening portion or through hole 91 for receiving the catheter 52 that extends from skin to the outside of the body of the patient P, and the opening portion 91 communicates with an open portion 92 that is a cutout part (slit-shaped) extending from the center to an outer edge portion of the receiving portion 25. Therefore, the base portions 20 and 21 can adhere to the skin of the patient P while receiving the catheter 52 that is in a state of puncturing the patient P in advance.

On the other hand, the second component 12 includes a holding portion 70 and a mounting portion 60.

The holding portion 70 is a portion that holds the catheters 51 and 52 or the connector 57 connected to the catheters 51 and 52.

The mounting portion 60 is a portion rotatably connected to a central portion 30 of the first component 11. The mounting portion 60 of the second component 12 is connected to be rotatable about the central portion 30. Therefore, the second component 12 can be positioned by being rotated to any position along an RR direction around the central portion 30, and a disposition direction of the catheters 51 and 52 on the body surface of the patient P can be freely changed with respect to the RR direction according to a procedure.

The first component 11 includes a catheter guide portion 40.

The catheter guide portion 40 is a portion having a role of bending the catheters 51 and 52, which extend from the receiving portion 25, toward the holding portion 70.

The catheter guide portion 40 is formed with a crush prevention portion 46. The crush prevention portion 46 is, for example, a portion having a curved surface whose bending radius or the like is set in order to prevent occurrence of a crush phenomenon (for example, a kink) of the catheter 51 or 52.

As shown in FIGS. 3 and 4, the first component 11 holds the catheters 51 and 52 that extend from the insertion port 200 to the outside of the body of the patient P while defining a bending angle of the catheters 51 and 52 in a state where the catheters 51 and 52 are inserted to the inside of the body of the patient P through the insertion port 200 as the puncture portion formed in the lumbar vertebrae S of the patient P.

The receiving portion 25 of the first component 11 includes, for example, the substantially columnar central portion 30.

The central portion 30 of the first component 11 is detachably fitted into the mounting portion 60 of the second component 12, and is rotatably connected to the second component 12.

The two base portions 20 and 21 are formed in different directions around the central portion 30 with the central portion 30 as a center.

The base portions 20 and 21 fix (adhere) the first component 11 to the body surface for the lumbar vertebrae S of the patient P, thereby stably and detachably fixing (adhering) the first component 11 to the lumbar vertebrae S.

Since the central portion 30 and the base portions 20 and 21 have relatively large thicknesses, an operator can easily stick or remove the base portions 20 and 21 and the central portion 30 to or from the body surface of the patient P while holding the base portions 20 and 21 and the central portion 30 by hand.

As shown in FIG. 3, adhesive portions 23 in close contact with the body surface of the patient P are preferably provided on back surfaces of the base portions 20 and 21.

The adhesive portion 23 is, for example, a gel adhesive substance, and can stably and detachably fix the first component 11 to the body surface of the patient P by fixing the back surfaces of the base portions 20 and 21 more closely to the body surface of the patient P.

When the adhesive portions 23 are not provided, the base portions 20 and 21 may be stably fixed to and in close contact with the body surface of the patient P by appropriately covering the base portions 20 and 21 using a separate surgical tape or the like.

As shown in FIG. 4, the base portions 20 and 21 extend in different directions from the central portion 30 with the central portion 30 as a center.

FIG. 4 shows that the base portions 20, 21 are elongated parts of the first component 11 that project away from a common upstanding part at the center of the first component. A shape of the base portions 20 and 21 is substantially rectangular, but is not particularly limited, and may be elliptical or semicircular.

By selecting a numerical value of an angle θ formed by the base portions 20 and 21 in any range, the two base portions 20 and 21 and the second component 12 cooperate with each other to stably dispose the first component 11 and the second component 12 on the body surface of the patient P by orienting the two base portions 20 and 21 and the second component 12 substantially equally in three directions.

That is, the catheter holder 10 constituted by the first component 11 and the second component 12 can perform holding on the body surface of the patient P so as to have substantially equal angles in three directions. Therefore, the catheter holder 10 can stably hold the spinal subarachnoid catheter 5 including the catheters 51 and 52 against the body surface of the patient P.

As shown in FIGS. 3 and 5, the catheter guide portion 40 provided in the substantially columnar central portion 30 of the first component 11 guides the catheters 51 and 52 or the connector 57 for the catheters 51 and 52, which extend through the insertion port 200 in the patient P to the outside of the body of the patient P, to a holding portion 70 side in the second component 12 and stably holds the catheters 51 and 52 and the connector 57.

The catheter guide portion 40 includes a plurality of catheter holding groove portions 41, 42, 43, 44, and 45, and the crush prevention portion 46.

As shown in FIGS. 3 and 4, the crush prevention portion 46 gently changes a direction from a vertical direction V to a horizontal direction H and guides the catheters 51 and 52 so as not to crush intermediate portions of the catheters 51 and 52 guided through the insertion port 200 in the patient P (so as not to cause a kink).

An intermediate portion 59 of the catheters 51 and 52 guided from the crush prevention portion 46 is selectively fitted into any one of the catheter holding groove portions 41, 42, 43, 44, and 45.

Accordingly, the intermediate portion 59 of the catheters 51 and 52 held in any one of the catheter holding groove portions 41, 42, 43, 44, and 45 is guided to the holding portion 70 side on the second component 12 while being changed in direction.

Depths of the catheter holding groove portions 41, 42, 43, 44, and 45 and a depth of the crush prevention portion 46 are preferably equal to or larger than an outer diameter of the catheter 52.

Accordingly, it is possible to ensure a state where the catheter 52 is embedded in any of the catheter holding groove portions 41, 42, 43, 44, and 45. Therefore, even when it is necessary to use a catheter made of a material harder than that of a silicone catheter, it is possible to avoid a risk that the catheters 51 and 52 are crushed by an external factor.

As shown in FIG. 4, the catheter holding groove portions 41, 42, 43, 44, and 45 are provided at different angles around a central axis CL of the central portion 30. In the shown example, the catheter holding groove portions 41, 42, 43, 44, and 45 are provided along a radial direction at regular angles around the central axis CL of the central portion 30.

Accordingly, by selectively fitting the intermediate portion 59 of the co-axial catheters 51 and 52 into any one of the catheter holding groove portions 41, 42, 43, 44, 45 to be held, a direction of the catheters 51 and 52 can be changed.

That is, since the catheter holding groove portions 41, 42, 43, 44, and 45 are provided at different angles around the central axis CL of the central portion 30, simply by selecting the catheter holding groove portions 41, 42, 43, 44, and 45 at any angle and fitting the catheters 51 and 52 thereto, the disposition direction of the catheters 51 and 52 on the body surface of the patient P can be easily and reliably changed according to a procedure, even when it is necessary to use a catheter made of a material harder than that of the silicone catheter.

Therefore, the catheters 51 and 52 are guided and fitted from any direction around the central axis CL, so that a positioning position in a circumferential direction (RR direction) in the central portion 30 shown in FIG. 4 can be selected and the catheters 51 and 52 can be held.

A set number of the catheter holding groove portions is not limited to the shown example and can be set freely.

As shown in FIG. 5, preferably, an adhesive portion 31 similar to the adhesive portions 23 of the base portions 20 and 21 is also provided on a back surface of the central portion 30.

Accordingly, by the adhesive portion 31 fixing the back surface of the central portion 30 in close contact with the body surface of the patient P, the adhesive portion 31 of the central portion 30 shown in FIG. 5 and the adhesive portions 23, 23 on the base portions 20 and 21 shown in FIG. 3 can cooperate with each other to detachably fix the first component 11 to the body surface of the patient P more stably.

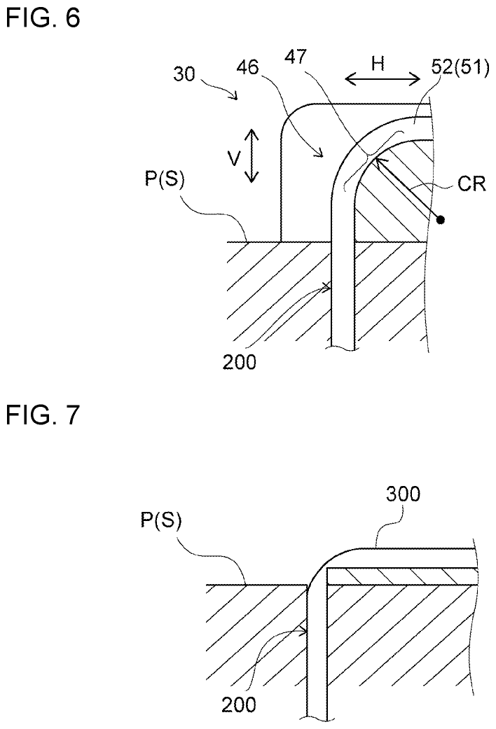

FIG. 6 is a longitudinal cross-sectional view showing a vicinity of the crush prevention portion 46 in the central portion 30 in the first embodiment.

The crush prevention portion 46 is formed in the central portion 30 so as to be cut along the horizontal direction (radial direction) H and the vertical direction V, and the crush prevention portion 46 is formed from a front surface to the back surface of the central portion 30.

The crush prevention portion 46 is formed of, for example, a relatively gentle circumferential portion 47 having a radius CR.

The circumferential portion 47 is preferably a ¼ circumferential portion. In this way, the crush prevention portion 46 includes the gentle circumferential portion 47, and therefore, the catheters 51 and 52 can be gently bent along the gentle circumferential portion 47 from the vertical direction V toward the horizontal direction H.

Therefore, even when it is necessary to use a catheter made of a material harder than that of the silicone catheter, the catheters 51 and 52 can be guided and extended from the insertion port 200 in the patient P to the outside of the body of the patient P while changing the direction from the vertical direction V to the horizontal direction H.

Accordingly, the crush prevention portion 46 can guide the catheters 51 and 52, which extends from the insertion port 200 in the patient P to the outside of the body, to any one of the catheter holding groove portions 41, 42, 43, 44, and 45 while appropriately providing the catheters 51 and 52 with an appropriate bending radius, without abruptly bending the catheters 51 and 52 from the vertical direction V to the horizontal direction H.

Therefore, the crush prevention portion 46 can prevent the catheters 51 and 52 from kinking (bending phenomenon) or completely breaking.

Therefore, even when it is necessary to use a catheter made of a material harder than that of the silicone catheter, a degree of freedom when the catheters 51 and 52 are routed in a procedure is improved, and handling of the catheters 51 and 52 is facilitated.

FIG. 7 shows an example of a normal use mode in which a catheter 300 is abruptly bent by 90 degrees and introduced into the insertion port 200 in the patient P as a comparative example with respect to the first embodiment.

In this normal use example, the catheter 300 is crushed in the vicinity of the insertion port 200, which is the puncture portion in the lumbar vertebrae, and is abruptly bent at a substantially right angle.

Therefore, since it is difficult to prevent a kink (bending phenomenon) of the catheter 300, a degree of freedom when the catheter 300 is routed in a procedure is small, and handling of the catheter 300 is difficult.

As shown in FIGS. 3 and 4, the second component 12 defines directionality of the catheters 51 and 52 in the RR direction on the body surface for the lumbar vertebrae S of the patient P.

As already described, the second component 12 includes the mounting portion 60 detachably mounted on the central portion 30 of the first component 11 and the holding portion 70. As shown in FIGS. 3 and 4, the mounting portion 60 of the second component 12 is a portion into which the central portion 30 of the first component 11 described above is fitted and which is rotatably fixed around the central axis CL of the central portion 30.

As shown in FIGS. 3 and 5, the mounting portion 60 includes a C-shaped portion 61 having a C shape circumscribing an outer periphery 33 of the central portion 30, and a cutout portion 62.

The cutout portion 62 is a distal portion of the second component 12 and is formed at a distal end of the C-shaped portion 61. The cutout portion 62 is provided in order to elastically deform the C-shaped portion 61 of the mounting portion 60 such that the C-shaped portion 61 slightly opens outward when the C-shaped portion 61 and the outer periphery 33 of the central portion 30 of the first component 11 are fitted to each other, which facilitates a fitting operation between the mounting portion 60 and the central portion 30.

As shown in FIGS. 3 and 5, the holding portion 70 of the second component 12 is, for example, a concave portion into which the catheter 52 or the connector 57 is detachably fitted and held.

Accordingly, the catheter 52 can be easily and reliably held by being fitted into the holding portion 70 that is a concave portion in a freely detachable manner.

Specifically, the holding portion 70 includes a first concave portion 71 that detachably fits and holds an outer peripheral portion of the co-axial catheter 52, and a second concave portion 72 that detachably fits and holds an outside of the connector 57 for the catheters 51 and 52.

Since a depth of the first concave portion 71 is larger than an external dimension of the catheter 52, a risk of the catheter 52 coming off from the first concave portion 71 is avoided.

The first concave portion 71 is connected to the second concave portion 72, and further, the second concave portion 72 includes fitting portions 73 and 74 into which the catheter 52 is laterally fitted.

The fitting portions 73 and 74 are formed in opposite directions, and are orthogonal to a longitudinal direction of the first concave portion 71.

A shape of a concave of the first concave portion 71 or the second concave portion 72 may be configured such that a wall is perpendicular to a bottom portion, or may be configured such that a width of an opening portion is smaller than a width of the bottom portion, and the width of the opening portion may be smaller than the outer diameter of the catheter. When the width of the opening portion is smaller than the outer diameter of the catheter, there is an advantage that it is difficult for the catheter to fall off.

### Fitting Portion 80

As shown in FIGS. 3 and 5, the first component 11 and the second component 12 are provided with the fitting portion 80.

The fitting portion 80 rotatably fits the mounting portion 60 of the second component 12 into the central portion 30 of the first component 11.

The fitting portion 80 may include, for example, a concave portion 81 (circumferentially extending groove) and a convex portion (projection portion or circumferentially extending projection) 82 detachably fitted into the concave portion 81.

The concave portion 81 is formed continuously or intermittently along a circular portion of the central portion 30, and has, for example, a semicircular or triangular cross section.

In contrast, the convex portion 82 is formed continuously or intermittently along the C-shaped portion 61 of the mounting portion 60, and has, for example, a semicircular or triangular cross section.

Simply by fitting the convex portion 82 into the concave portion 81 in the fitting portion 80, the second component 12 can be reliably connected to the first component 11 in a rotatable manner along the RR direction with a simple structure.

By forming the concave portion 81 around the central portion 30 of the first component 11 and forming the convex portion 82 on the mounting portion 60 of the second component 12, the second component 12 can be reliably connected to the first component 11 in a rotatable manner with a simple structure.

In contrast to the shown example, the convex portion may be formed on an outer peripheral surface of the central portion 30, and the concave portion may be formed in an inner peripheral surface of the mounting portion 60.

As shown in FIG. 3, preferably, an adhesive portion 29 is provided on a back surface of the second component 12 in a similar manner as the adhesive portions 23 on the base portions 20 and 21.

Accordingly, the second component 12 can be detachably fixed to and in close contact with the body surface of the patient P in a similar manner as the first component 11. Even when it is necessary to use a catheter made of a material harder than that of the silicone catheter, the catheters 51 and 52 can be reliably and stably held on the body surface of the patient P without displacement.

### Usage Example of Catheter Holder 10

Next, a usage example of the catheter holder 10 will be described with reference to the drawings.

When the patient P is treated for a brain disease, the insertion port 200, which is a puncture portion, is formed in advance in the vicinity of the lumbar vertebrae S of the patient P as shown in FIG. 3.

First, as shown in FIG. 3, the two base portions 20 and 21 of the first component 11 are positioned and stuck in the vicinity of the insertion port 200 in the body surface of the patient P.

At this time, as shown in FIG. 3, the first component 11 is stuck (adhered) to the body surface of the patient P such that a position of the crush prevention portion 46 at the central portion 30 corresponds to the vicinity of the insertion port 200.

Next, as shown in FIG. 3, the central portion 30 of the first component 11 is mounted in the mounting portion 60 of the second component 12, so that the outer periphery 33 of the central portion 30 is fitted into the C-shaped portion 61 of the mounting portion 60.

Since the convex portion 82 on the mounting portion 60 fits into the concave portion 81 in the central portion 30 at the same time as the fitting operation, the second component 12 is less likely to come off from the first component 11, and the second component 12 is rotatably held around the central axis CL of the central portion 30.

As shown in FIGS. 2A and 2B, a disposition direction when the second component 12 is mounted on the first component 11 can be changed according to the convenience of the procedure, and a set angle of the second component 12 can be changed with respect to the first component 11 around the central axis CL.

That is, the angle of the second component 12 can be easily changed with respect to the first component 11 around the central axis CL in the RR direction.

In the example shown in FIG. 2A, the intermediate portion 59 of the catheters 51 and 52 is selectively fitted and held in the catheter holding groove portion 43. However, in the example shown in FIG. 2B, the intermediate portion 59 of the catheters 51 and 52 is selectively fitted and held in the catheter holding groove portion 44.

In this way, the second component 12 can be freely set in an extending direction of the catheters 51 and 52 according to the convenience of the procedure.

After the disposition direction of the second component 12 is adjusted to a state suitable for the procedure, the catheter holder 10 is fixed to the body surface of the patient P in an appropriate disposition state by, for example, sticking or fixing an adhesive from the catheter holder 10 to the body surface.

In this case, the catheters 51 and 52 are guided to the outside of the body of the patient P though the insertion port 200 in the patient P shown in FIG. 3, and as shown in FIG. 6, the direction of the catheters 51 and 52 can be changed by bending from the vertical direction V toward the horizontal direction H along the gentle circumferential portion 47 of the crush prevention portion 46.

Accordingly, the crush prevention portion 46 can change the direction of the catheters 51 and 52 guided through the insertion port 200 toward any one of the catheter holding groove portions 41, 42, 43, 44, and 45 while maintaining an appropriate bending radius without abruptly bending the catheters 51 and 52 from the vertical direction V to the horizontal direction H.

Therefore, it is possible to prevent the catheters 51 and 52 from kinking (bending phenomenon) or completely breaking. Therefore, even when it is necessary to use a catheter made of a material harder than that of the silicone catheter, the degree of freedom when the catheters 51 and 52 are routed in the procedure is improved, and the handling of the catheters 51 and 52 is facilitated.

The catheter holder 10 is divided into two components, i.e., the first component 11 and the second component 12, to share a role, and thus it is possible to prevent a phenomenon in which the catheters 51 and 52 are kinked.

The second component 12 can move with respect to the first component 11 in the RR direction, which is the radial direction, on the body surface for the lumbar vertebrae S. Therefore, the catheters 51 and 52 can be stably fixed on the body surface of the patient P by selecting a disposition direction, and even when it is necessary to use a catheter made of a material harder than that of the silicone catheter, a degree of freedom in a direction in which the catheter is routed is improved, and the catheter can be freely positioned and is easily handled during a procedure.

Next, a second embodiment of the catheter will be described.

A catheter holder 110 according to the second embodiment has a similar structure as the catheter holder 10 according to the first embodiment, similar parts are denoted by the same reference signs and a detailed description of such parts is not repeated. The following description focuses on differences.

### Second Embodiment

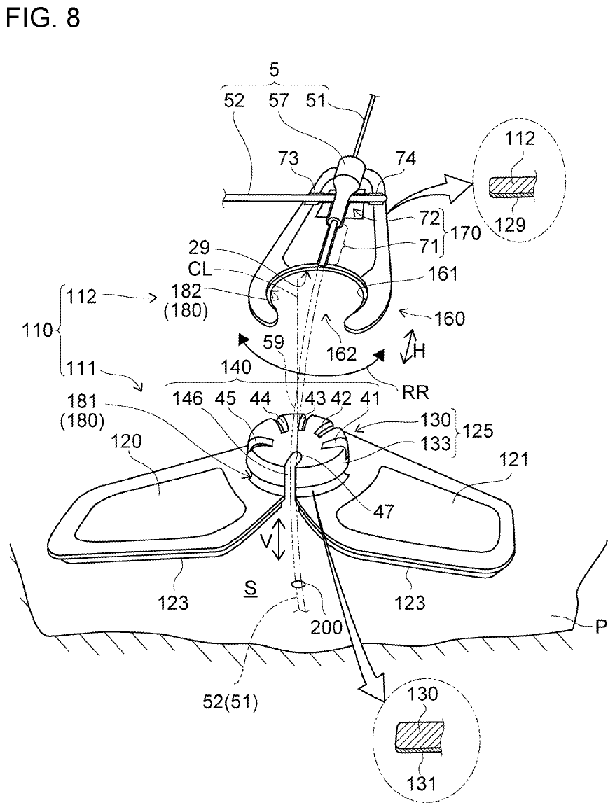

FIG. 8 is an exploded perspective view showing the second embodiment of the catheter.

As shown in FIG. 8, the catheter holder 110 includes a first component 111 and a second component 112.

The first component 111 preferably includes base portions 120 and 121 and a receiving portion 125.

The base portions 120 and 121 can adhere to, for example, the body surface for the lumbar vertebrae S of the patient P.

The receiving portion 125 is a portion capable of receiving portions of the catheters 51 and 52 disposed in the inside of the body of the patient P that extend to the outside of the body.

The second component 112 includes a holding portion 170 and a mounting portion 160.

The holding portion 170 holds the catheters 51 and 52 or the connector 57 connected to the catheters 51 and 52.

The mounting portion 160 is a portion rotatably connected to the first component 111.

The first component 111 or the second component 112 includes a catheter guide portion 140.

The catheter guide portion 140 is a portion having a role of bending the catheters 51 and 52, which extend from the receiving portion 125, toward the holding portion 170.

The catheter guide portion 140 is formed with a crush prevention portion 146.

The crush prevention portion 146 has, for example, a curved surface whose bending radius or the like is set in order to prevent occurrence of a kink of the catheters 51 and 52.

As shown in FIG. 8, the base portions 120 and 121 of the first component 111 have a role of stably fixing the first component 111 to the lumbar vertebrae S of the patient P by adhering the first component 111 to the body surface for the lumbar vertebrae S.

A central portion 130 and the base portions 120 and 121 shown in FIG. 8 are formed relatively thinner than the base portions 20 and 21 in the first embodiment shown in FIG. 4. Accordingly, a weight and a size of the first component 111 can be reduced.

Adhesive portions 123 in close contact with the body surface of the patient P are preferably provided on back surfaces of the base portions 120 and 121.

The adhesive portion 123 is, for example, a gel substance, and can stably and detachably fix the first component 111 to the body surface of the patient P by fixing the back surfaces of the base portions 120 and 121 in close contact with the body surface of the patient P.

When the adhesive portions 123 are not provided, the base portions 120 and 121 may be stably fixed to and in close contact with the body surface of the patient P by appropriately covering the base portions 120 and 121 using a separate surgical tape or the like.

The base portions 120 and 121 extend in different directions from the central portion 130 with the central portion 130 as a center.

The base portions 120 and 121 each have a substantially rectangular shape. The catheter holder 110 constituted by the first component 111 and the second component 112 can be held on the body surface of the patient P so as to have substantially equal angles in three directions. Therefore, even when it is necessary to use a catheter made of a material harder than that of the silicone catheter, the catheter holder 110 can stably hold the catheters 51 and 52 and the connector 57 against the body surface of the patient P.

Since the second component 112 is formed thinner than the second component 12 in the first embodiment shown in FIG. 3, a thickness and a weight can be reduced.

The central portion 130 of the first component 111 is connected to the mounting portion 160 of the second component 112 such that the mounting portion 160 of the second component 112 is rotatable about the central axis CL of the central portion 130.

The mounting portion 160 of the second component 112 includes a C-shaped portion 161 circumscribing an outer periphery 133 of the central portion 130, and a cutout portion 162.

The cutout portion 162 is provided in order to elastically deform the C-shaped portion 161 of the mounting portion 160 such that the C-shaped portion 161 slightly opens when the C-shaped portion 161 and the outer periphery 133 of the central portion 130 of the first component 111 are fitted to each other. Accordingly, the C-shaped portion 161 and the outer periphery 133 are brought into close contact with each other, and a fitting operation is facilitated.

As shown in FIG. 8, the first component 111 and the second component 112 are provided with a fitting portion 180.

The fitting portion 180 rotatably connects the mounting portion 160 of the second component 112 to the central portion 130 of the first component 111.

The fitting portion 180 includes a concave portion 181 in the central portion 130 and a convex portion (projection portion) 182 on the mounting portion 160 detachably fitted into the concave portion 181.

Preferably, an adhesive portion 131 is provided on a back surface of the central portion 130 of the first component 111, and an adhesive portion 129 is provided on a back surface of the second component 112 in a similar manner as the adhesive portions 123 on the base portions 120 and 121.

Accordingly, by fixing the first component 111 and the second component 112 in close contact with the body surface of the patient P, even when it is necessary to use a catheter made of a material harder than that of the silicone catheter, both the first component 111 and the second component 112 can be stably and detachably fixed to the body surface of the patient P.

The catheter holder 110 is divided into two components, i.e., the first component 111 and the second component 112, and thus it is possible to prevent a phenomenon in which the catheters 51 and 52 are kinked or completely bent in, for example, a procedure using the spinal subarachnoid catheter 5.

The second component 112 can operate on the body surface for the lumbar vertebrae S with respect to the catheters 51 and 52 in the RR direction. The catheters 51 and 52 and the connector 57 can be stably fixed to the body surface of the patient P, the degree of freedom in the direction in which the catheter is routed is improved, and the catheter can be freely positioned and is easily handled during a procedure.

Various embodiments of the catheter have been described above. However, the invention is not limited to the above embodiments, and various modifications can be made without departing from the scope of the claims. A part of the configuration of the above embodiments may be omitted or may be freely combined as different from the above.

The shown brain disease treatment system 1 is an example of a medical system in which the catheter holder 10 is used. However, the medical system to which the catheter holder 10 according to the embodiment of the invention can be applied is not limited thereto, and the catheter holder 10 can be applied to medical systems in other regions and fields.

For example, in the shown first component 11, a size of the base portions 20 and 21 may be reduced, or the base portions 20 and 21 may be omitted. Accordingly, the first component 11 and the catheter holder 10 can be reduced in size and weight.

Although FIG. 3 shows a configuration in which the catheter guide portion 40 is provided in the first component 11, the catheter guide portion 40 may be provided in the second component 12, and the second component 12 including the catheter guide portion 40 may be rotatably connected to the first component 11.

The detailed description above describes embodiments of a catheter and an operational method representing examples of the new catheter and operational method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter holder comprising:
a first component including a base portion configured to be adhered to a body surface of a body of a patient and a receiving portion configured to receive a portion of a catheter that is disposed inside the body of the patient and that extends to outside of the body; a second component including a holding portion configured to hold the catheter and/or a connector connected to the catheter, the second component being rotatably connected to the first component;
the first component including a catheter guide portion configured to bend the catheter, which extends from the inside of the body of the patient, toward the holding portion; and
the catheter guide portion being formed with a crush prevention portion configured to prevent the catheter from being crushed.

2. The catheter holder according to claim 1, wherein
the first component includes a central portion to which the second component is rotatably connected, the base portion being formed around the central portion, and
the catheter guide portion is formed in the central portion.

3. The catheter holder according to claim 2, wherein the base portion is a first base portion, and further comprising a second base portion, the first and second base portions extending in different directions away from the central portion.

4. The catheter holder according to claim 1, wherein
the second component includes a mounting portion detachably mounted on the central portion of the first component, and the central portion is connected to the mounting portion of the second component such that the mounting portion of the second component is rotatable about the central portion.

5. The catheter holder according to claim 1, wherein the catheter guide portion includes a catheter holding groove into which an intermediate portion of the catheter is positionable in the central portion.

6. The catheter holder according to claim 5, wherein the catheter holding groove is one of a plurality of catheter holding grooves provided in the central portion, and the catheter holding grooves being provided at different angles with respect to a center line of the central portion.

7. The catheter holder according to claim 1, wherein the holding portion is concave groove configured to detachably receive and hold the catheter.

8. The catheter holder according to claim 4, further comprising:
a fitting portion configured to rotatably connect the mounting portion of the second component to the central portion of the first component,
the fitting portion including a concave portion and a convex portion fitted into the concave portion.

9. The catheter holder according to claim 8, wherein the fitting portion includes a groove as the concave portion formed around the central portion of the first component, and a projection as the convex portion formed in the mounting portion of the second component and fitted into the groove.

10. The catheter holder according to claim 4, wherein the mounting portion of the second component is C-shaped and is fitted to the central portion of the first component so that the second component is rotatable about the central portion of the first component.

11. The catheter holder according to claim 1, wherein an opening portion configured to receive the catheter that extends from the body surface of the patient to the outside of the body is provided at a center of the receiving portion, the receiving portion has a cutout open portion at an outer edge portion, and the opening portion and the open portion communicate with each other.

12. A catheter holder comprising:
a first component including a receiving portion configured to receive a portion of a catheter that is disposed inside a body of a patient and that extends to outside the body;
a second component including a holding portion configured to hold the catheter and/or a connector connected to the catheter, the second component being rotatably connected to the first component,
the first component or the second component including a catheter guide portion configured to bend the catheter, which extends from the receiving portion, toward the holding portion, and
the catheter guide portion being formed with a crush prevention portion configured to prevent the catheter from being crushed.

13. A catheter holder in combination with a catheter to be mounted on a body of a patient to hold the catheter when the catheter is disposed inside the patient's body and extends to outside of the patient's body, the catheter holder comprising:
a first component that includes a receiving portion and a body surface fixing base portion, the body surface fixing base portion projecting away from the receiving portion as seen in plan view, the body surface fixing base portion being configured to be fixed to a body surface of the patient's body, the receiving portion having a surface configured to face the body surface of the patient when the body surface fixing base portion is fixed to the body surface of the patient's body, the receiving portion including a through hole that extends through the receiving portion so that the through hole intersects the surface of the receiving portion, the through hole in the receiving portion being configured to receive a portion of the catheter passing through the through hole and extending outside the patient's body;
a second component including a mounting portion and a holding portion, the holding portion projecting away from the mounting portion as seen in the plan view and being configured to hold the portion of the catheter extending outside the patient's body;
the mounting portion being mounted on the receiving portion by virtue of the receiving portion and the mounting portion including engaging parts that are in engagement with one another in a manner that allows the mounting portion to be rotated relative to the receiving portion; and
the receiving portion of the first component including a crush prevention portion constituted by a curved circumferential portion that merges with the through hole so that the portion of the catheter passing through the through hole passes over the curved circumferential portion and is led to the holding portion of the second component.

14. The catheter holder in combination with the catheter according to claim 13, wherein the engaging portions include a circumferentially extending projection and a circumferentially extending groove, the circumferentially extending projection being positioned in the circumferentially extending groove.

15. The catheter holder in combination with the catheter according to claim 13, wherein the mounting portion is a C-shaped mounting portion as seen in the plan view, the C-shaped mounting portion including a cutout portion that communicates with the through opening.

16. The catheter holder in combination with the catheter according to claim 13, wherein the holding portion includes a groove that receives the portion of the catheter extending outside the patient's body.

17. The catheter holder in combination with the catheter according to claim 13, wherein the receiving portion includes a plurality of circumferentially spaced apart catheter holding grooves, each of the catheter holding grooves being radially oriented relative to the through hole as seen in the plan view.

18. The catheter holder in combination with the catheter according to claim 13, wherein the surface of the body surface fixing base portion of the first component includes an adhesive to adhere the body surface fixing base portion to the surface of the patient's body.

19. The catheter holder in combination with the catheter according to claim 13, wherein the body surface fixing base portion of the first component is a first body surface fixing base portion, the first component including a second body surface fixing base portion that projects away from the receiving portion as seen in the plan view, the first and second body surface fixing base portions projecting away from the receiving portion in different directions.

20. The catheter holder in combination with the catheter according to claim 13, wherein
the catheter is a spinal subarachnoid catheter that includes
a supply catheter configured to supply a treatment liquid to a target lesion of the patient,
a discharge catheter disposed coaxially with the supply catheter and configured to discharge the liquid from the target lesion to the outside of the body, and
an interlock component configured to interlock the supply catheter and the discharge catheter, and
the holding portion of the second component is configured to detachably fit and hold a part of the spinal subarachnoid catheter and the interlock component.
